(19) United States
(12) Patent Application Publication
(10) Pub. No.: US 2008/0275489 A1
(43) Pub. Date: Nov. 6, 2008

(54) REMOVABLE MEDICAL FILTER

(76) Inventors: Thomas Frank Kinst, Hillsborough, NJ (US); James A. Fleming, Bethlehem, PA (US)

Correspondence Address: RONALD L. HOFER, 122 LINDBERGH LANE, MOORESVILLE, NC, 28117, US

(21) Appl. No.: 11/742,706

(22) Filed: May 1, 2007

(51) Int. Cl. A61M 29/00

(52) U.S. Cl. 606/200

(57) ABSTRACT

A medical filter intended for placement inside a tubular body passage such as a blood vessel of a patient, the medical filter having an end member at one end of the longitudinal axis of the filter. The end member supports a retrieval element and a plurality of struts defining a first filter cone proximate to the end member and a second filter cone distal from the end member.

### BACKGROUND AND SUMMARY OF THE INVENTION

The present invention relates to medical filters which are intended to be placed inside a blood vessel or other body passage for the purpose of intercepting thrombus or particles.

Medical filters, including vena cava filters, are emplaced inside blood vessels or other body passages to trap unwanted particles. When used in a blood vessel, a medical filter should be effective to entrap thrombus, clots or other dangerous coagulations or particulate matter in the blood while allowing free flow of blood through the filter in the vessel. While in some cases it is desirable to leave a medical filter in place indefinitely, in other cases it is desirable to retrieve the filter after it has been in place for a period of time. Such retrieval can be difficult. For example, when a vascular filter is placed within a vessel of the human body for any significant length of time, the inner lining of the vessel will grow around the portion of the device that is in contact with the tissue and encapsulate it. Tissue does not attach itself directly to materials such as Nitinol an stainless steel and it is known to make medical filters out of materials. However, depending upon the design of the filter, removal can still be difficult if there is mechanical interference between the tissue and elements of the filter. Corners, loops or other features of the filter must not interfere with tissue when the filter is retrieved in order for the retrieval to be atraumatic.

Another important feature desirable in a medical filter is that it be resistant to migration. This is particularly the case with vascular filters. Thus, it is highly desirable that the filter resist movement in a vessel after it has been deployed yet be readily retrievable without tearing the tissue of the vessel. Of course, there are several other characteristics which are generally desirable in medical filters. For example, the filter should be of a design which can be deployed in a proper position in the blood vessel or body passage with minimal trauma to the patient and of a design which does not become entangled during its deployment. It is also desirable that the filter be adapted to center in the vessel and to properly adjust to the size of the vessel and that the filter remain effective during its time in place, i.e., resist occlusion.

Although vascular and other medical filters are known in the art and have been found to be effective, the requirements of such filters can be high and there remains room for improved designs. In particular, there remains room for improved filters which exhibit the above-mentioned characteristics and yet can be economically manufactured. Accordingly, the present invention provides a medical filter which is suitable for placement in a blood vessel or body passage for an extended duration without migrating in the vessel or body passage and yet can be removed or retrieved with minimal trauma to the tissue of the blood vessel or body passage. Furthermore, the filter of this invention is effective for long duration and is resistant to occlusion.

In accordance with the present invention, a preferred embodiment of the present invention is an elongated medical filter which has a longitudinal axis, is radially expandable and compressible and is intended for placement inside a tubular body passage, such as a blood vessel, of a patient. In expanded form, the medical filter comprises:
(A) an end member at one end of the longitudinal axis of the filter with a retrieval element;
(B) a plurality of struts extending generally radially outwardly and axially from said end member, each of said struts having a middle segment extending generally in a cylindrical plane about said longitudinal axis and an end segment extending generally radially inwardly and axially with free ends spaced from said longitudinal axis.
   In one preferred embodiment of the present invention, the middle segments of the struts extend generally parallel to each other and to the longitudinal axis of the filter. In an alternative preferred embodiment of the present invention, the middle segments of the struts are in the plane of the cylinder and extend in a spiral fashion. In further alternative preferred embodiment of the present invention, the middle segments of the struts are in the plane of the cylinder and extend in a spiral fashion while the end segments also extend in a spiral fashion. In a still further alternative embodiment of the present invention, the middle segments of the struts extend parallel to each other and to the longitudinal axis and the end segments extend in a spiral fashion.

Further understanding of the present invention will be had from the following description taken in conjunction with the accompanying drawings and appended claims.

### BRIEF DESCRIPTION OF THE DRAWINGS

FIG. 1 is a side elevational view of a preferred embodiment of a medical filter of the present invention shown in expanded form;

FIG. 2 is an end view of the preferred embodiment of FIG. 1 as viewed from the right in FIG. 1;

FIG. 3 is a side elevational view of an alternative preferred embodiment of a medical filter of the present invention shown in expanded form;

FIG. 4 is an end view of the alternative preferred embodiment of FIG. 3 as viewed from the left in FIG. 3;

FIG. 5 is a side elevational view of further alternative preferred embodiment of a medical filter of the present invention shown in expanded form; and

FIG. 6 is an end view of the further alternative preferred embodiment of FIG. 5 as viewed from the left in FIG. 5.

### DETAILED DESCRIPTION OF THE PREFERRED EMBODIMENTS

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the preferred embodiments of the present invention are described in conjunction with a blood vessel but the preferred embodiments may also be used in other body passages.

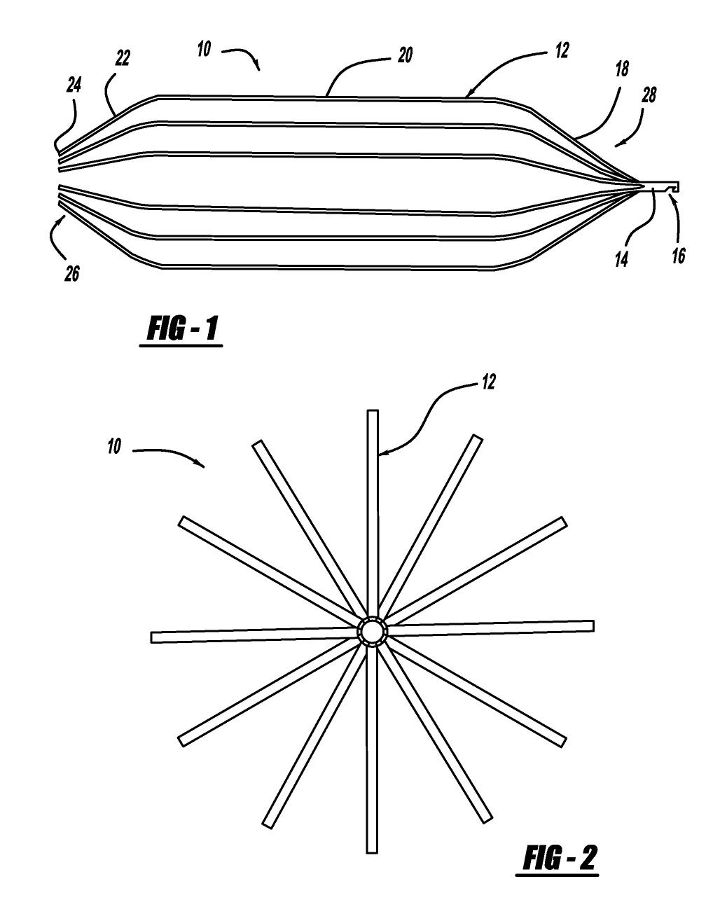

Now referring to FIGS. 1 and 2, a preferred embodiment of a medical filter of the present invention is shown and indicated generally by the numeral 10. Filter 10 is made of a resilient material, preferably a shape memory material, which tends to expand to the form illustrated in FIGS. 1 and 2 but can be compressed radially to a smaller diameter to be carried in the lumen of a suitable delivery catheter (not shown) as is conventional in the art.

Generally speaking medical filter 10 is radially expandable and compressible and is intended for placement inside a tubular body passage, such as a blood vessel, of a patient. It is contemplated that medical filter 10 will be compressed and placed into the lumen of a delivery catheter and then delivered to the desired site in a blood vessel or other body cavity whereupon filter 10 will be ejected from the catheter and allowed to self expand in position if the vessel or cavity. FIGS. 1 and 2 illustrate filter 10 in expanded form. In expanded form, medical filter 10 broadly comprises a plurality of struts 12 with ends attached or otherwise secured to an end member 14 which is on the longitudinal axis of filter 10.

End member 14 can be formed as one piece with struts 12 or can be a collar or ring to which struts 12 are welded or otherwise attached as by adhesive means. Alternatively, end member 14 can be formed from the ends of struts 12 themselves being welded or otherwise secured to each other. End member 14 carries a retrieval element 16 which can be a retrieval hook or the like. Retrieval element 16 can be a single hook as shown in FIG. 1 or can have a T-shape with twin hooks or any other suitable shape which can operatively interact with a snare device or any other suitable element which will operatively interact with a retrieval means. It will be appreciated by those skilled in the art that hooks and snares are well-known and that the particular retrieval means employed is subject to variation within the spirit of the present invention.

Struts 12 each have a first filter segment 18 which extends generally radially outwardly and axially from end member 14. Each first filter segment 18 extends to a middle segment 20 which extends generally axially and parallel to the longitudinal axis of filter 10. Each middle segment 20 extends to a generally radially inwardly and axially extending filter segment 22 with free end 24. As shown in the figures, the plurality of struts 12 are spaced radially about the longitudinal axis with middle segments 20 in the plane of a cylinder centered on the longitudinal axis. Free ends 24 of struts 12 are spaced from each other and from the longitudinal axis of filter 10 and are unattached to each other or any other element of filter 10.

Filter 10 may be made of any suitable material using a variety of methods. Nitinol is a preferred material but elgiloy, cobalt chromium, stainless steel or suitable plastic are examples of other materials that may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter. Thus, the material must also be sufficiently resilient to accomplish both compression in the delivery catheter and expansion upon ejection from the catheter.

Suitable methods of manufacture of medical filter 10 include cutting a pattern into a tube to enable expansion of the tube into the desired body and struts. Another suitable method is forming the struts and body from separate strips or wires and then joining the respective parts together by suitable methods which are well known in the art.

In use in a blood vessel or the like, it is contemplated that filter 10 will be preferably oriented with its “open” end 26 facing downstream. Thus, clots or other unwanted thrombus or other material to be filtered will first encounter the “closed” end 28 of filter 10 and tend to be forced radially outwardly toward the wall of an associated blood vessel or wall of a body passage where they will tend to become trapped. A further filter basket is provided by the radially inwardly extending filter segments 22 of struts 12.

The free ends 24 of struts 12 facilitate removal of filter 10 from an associated vessel or body passage. Filter 10 has struts with no closed elements which will be in contact with a vessel wall and is thus designed for long term retrievability. Thus, although filter 10 is suitable for permanent placement in a vessel or body passage, filter 10 may be readily removed or retrieved if retrieval is desired. Retrieval of filter 10 may be accomplished by means of a conventional retrieval catheter which may be inserted from the appropriate direction to approach the end of filter 10 having retrieval element 16. After snaring retrieval element 16 with a snare, filter 10 is drawn into the lumen of the retrieval catheter and retrieved in a conventional manner. It will be appreciated that the free ends 24 of struts 12 allow removal of struts 12 from any tissue that may have grown around them with minimal trauma.

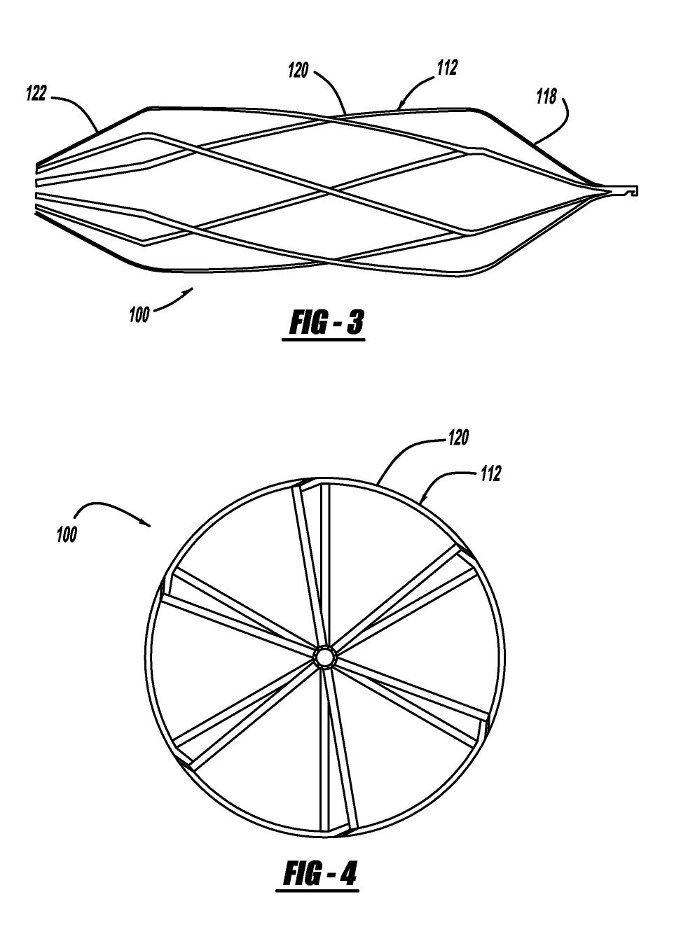

Now referring to FIGS. 3 and 4, an alternative preferred embodiment of the present invention is shown and indicated generally by the numeral 100. Medical filter 100 is of a material and construction generally analogous to medical filter 10 and can be made by substantially the same method of manufacture. Medical filter 100 differs from medical filter 10 in the direction of orientation of struts 112. As best shown in FIG. 3, struts 112 are analogous to struts 12 with respect to segments 18 and 22, but middle segments 120 do not extend generally parallel to each other. In the case of medical filter 100, middle segments 120 of struts 112 extend in a twisted, spiral pattern. Middle segments 120 form a cylindrical shape about the longitudinal axis of filter 100.

As is best shown in FIG. 3, medical filter 100 has a filter basket defined by first filter segments 118 and a second filter basket defined by second filter segments 122. The spiral pattern of segments 118 and 122 can be adjusted to increase or decrease the interference that the cone shaped baskets provide to the flow path through filter 100 and thereby increase or decrease the filtering efficiency of filter 100.

The spiral design of middle segments 120 provides increased resistance to migration of medical filter 100 when it is in place in a vessel or the like. The exact spiral pattern of middle segments 120 can be varied to increase or decrease the migration resistance of filter 100.

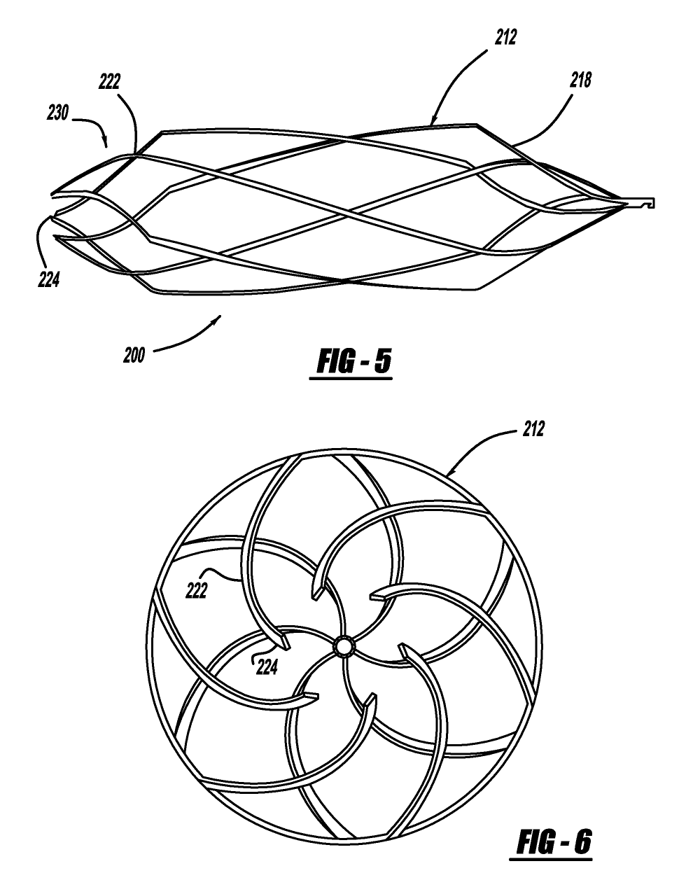

Now referring to FIGS. 5 and 6 a further alternative preferred embodiment of the present invention is shown and indicated generally by the numeral 200. Medial filter 200 is generally analogous to medical filter 100 and has first filter segments 218 but has a second filter basket defined by segments 222 of struts 212 which extend generally radially inwardly and axially but in a spiral manner to define a second filter segment 230. Free ends 224 of struts 212 are spaced from each other and from the longitudinal axis of filter 10 and are unattached to each other or any other element of filter 200.

The spiral relationship of segments 222 provides a cone shaped basket and increases the density of the wires or struts in the flow path to increase the filtering efficiency of medical filter 200. The pitch of the spiral pattern can be varied to further increase or decrease the filtering efficiency. The spiral pattern of segments 222 only slightly, if at all, increases resistance to retrieval of medical filter 200 from an associated vessel or body passage.

While preferred embodiments of the present invention have been specifically described above, it will be appreciated by those skilled in the art that the present invention is subject to variations and modifications. For example, the filter may be cut from a single tube and have an end member which is integral, i.e., one piece, with the struts. The filter may be tube-based or wire based or a combination. The filter baskets may be modified by modifying the number of struts and/or their pattern. The retrieval element may be modified by changing the angle of the hook and/or by modifying the number of tines for engaging the retrieval snare. These and other modifications are contemplated to be within the scope of the present invention which is intended to be limited only by the following claims.

What is claimed is:

1. An elongated, radially expandable and compressible medical filter with a longitudinal axis, intended for placement inside a tubular body passage such as a blood vessel of a patient, the medical filter comprising in expanded form:
(A) an end member at one end of the longitudinal axis of the filter with a retrieval element;
(B) a plurality of struts with first segments extending generally radially outwardly and axially from said end member, each of said struts having a middle segment extending generally in a cylindrical plane about said longitudinal axis and an end segment extending generally radially inwardly and axially with free ends spaced from said longitudinal axis.

2. The medical filter of claim 1 wherein said middle segments of said struts extend generally parallel to each other and to the longitudinal axis of the filter.

3. The medical filter of claim 1 wherein said middle segments of said struts are in the plane of a cylinder and extend in a spiral manner.

4. The medial filter of claim 3 wherein said first segments extend in a spiral manner.

5. The medical filter of claim 4 wherein said end segments extend in a spiral manner.

6. The medical filter of claim 2 wherein said end segments extend in a spiral manner.

7. The medical filter of claim 1 wherein said retrieval element is a hook.

8. The medical filter of claim 1, wherein said filter is comprised of Nitinol.

9. The medical filter of claim 1 wherein said filter is comprised of stainless steel.

10. The medical filter of claim 1 wherein said struts are comprised of wire.

11. The medical filter of claim 1 wherein said struts are cut from tubing.

12. A radially expandable and compressible, elongated medical filter having a longitudinal axis, said filter intended for placement inside a tubular body passage of a patient and comprising in expanded form:
(A) an end member at one end of the longitudinal axis of the filter with a retrieval element;
(B) a plurality of struts with first segments extending generally radially outwardly and axially from said end member to define a cone shaped first filter basket, each of said struts having a middle segment extending generally in a cylindrical surface about said longitudinal axis and an end segment extending generally radially inwardly and axially with free ends spaced from said longitudinal axis to define a cone shaped second filter basket.

13. The medical filter of claim 12 wherein said middle segments of said struts extend generally parallel to each other and to the longitudinal axis of the filter.

14. The medical filter of claim 12 wherein said middle segments of said struts are in the surface of a cylinder and extend in a spiral manner.

15. The medical filter of claim 12 wherein said first segments extend in a spiral manner.

16. The medical filter of claim 12 wherein said end segments extend in a spiral manner.

17. The medical filter of claim 15 wherein said end segments extend in a spiral manner.

18. The medical filter of claim 13 wherein said filter is comprised of Nitinol.

19. The medical filter of claim 13 wherein said filter is comprised of stainless steel.

20. The medical filter of claim 1 wherein said legs are comprised of wire.

21. The medical filter of claim 1 wherein said legs are cut from tubing.
